(19) United States
(12) Patent Application Publication
(10) Pub. No.: US 2002/0022048 A1
(43) Pub. Date: Feb. 21, 2002

(54) Composite wafer for controlled drug delivery

(76) Inventors: Lev E. Bromberg, Swampscott, MA (US); Phillip M. Friden, Bedford, MA (US)

Correspondence Address: CLARK & ELBING LLP, 176 FEDERAL STREET, BOSTON, MA, 02110-2214, US

(21) Appl. No.: 09/865,864

(22) Filed: May 25, 2001

Related U.S. Application Data
(60) Provisional application No. 60/207,663, filed on May 26, 2000.

(51) Int. Cl. A61K 47/00; A61K 9/28; A61K 9/68; A61K 9/70; A61F 13/00

(52) U.S. Cl. 424/439; 424/441; 424/449

(57) ABSTRACT

This invention relates to a completely bioerodable film that adheres to mucosal surfaces and provides sustained pharmaceutical delivery for several weeks.

This application claims priority from U.S. Patent Application No. 60/207,663, filed on May 26, 2000.

### BACKGROUND OF THE INVENTION

Drug delivery systems comprising small particles can be administered into the desired oral cavity or periodontal pocket with relative ease, but they do not always satisfy the demand for a bioerodable implant. Because they are particles, they do not form a continuous film or solid implant with the structural integrity needed for certain prostheses. When inserted into the mouth, periodontal pocket, or other lumenal cavity where there is considerable fluid flow, these small particles, microspheres, or microcapsules are poorly retained because of their size and discontinuous nature. Moreover, due to the microscopic size of the particles of bioerodable polymer, the rate of release does not provide a sustainable delivery lasting several weeks.

Biodegradable drug delivery systems capable of formation of films applied below gum line are described in U.S. Pat. No. 5,945,115 and U.S. Pat. No. 5,990,194. Therein, a biodegradable polymer is provided for use in providing syringeable, in-situ forming, solid biodegradable implants. The polymer is administered in liquid form and cures to form the implant in-situ. However, the use of organic solvents foreign to the human body is implied. Moreover, precipitates formed upon phase separation of the water-insoluble thermoplastic biodegradable materials upon contacting water are not bioadhesive by virtue of their hydrophobicity. This limits the retention time of such implants. Therefore, there exists a need for a method and composition, which provides a bioerodable, macroscopic polymeric structure useful in overcoming the above-described limitations.

Biodegradable wafers for use in the delivery of antimicrobial agents to the periodontal pocket have been reported. A cross-linked gelatin polymer wafer, available under the trademark PerioChip® from Astra Pharmaceuticals, LP, Westborough, Mass., has been shown to reduce subgingival bacteria when inserted into the periodontal pocket and to release chlorhexidine, an antimicrobial agent, for seven to ten days which is considered too short a time period for treatment of many periodontal diseases. Only a small improvement in periodontal pocket depth was observed.

The presence of a plasticizer within a sustained release composition is known to advance or speed up the release of bioactive material by the sustained release polymer. Plasticizers have been used to enhance the delivery of drugs from diffusional therapeutic systems. Water-insoluble liquid plasticizers are used to “soften” the polymer and cause its diffusion coefficient to increase, thereby enhancing the diffusion of nonionic drugs. Water-soluble plasticizers are used to create a water-swollen microporous structure, by leaching slowly from the polymer, to make the composition more permeable to drugs.

Thus, the prior art drug delivery vehicles do not provide adequate retention, bioadhesion and drug release characteristics for prolonged therapeutic delivery in moist environments like a periodontal pocket. U.S. Pat. No. 6,159,498, for example, describes the advantages film delivery systems provide over bioadhesive gels and tablets, and describes a bioerodable film device. However, the maximum residence time of film described in the '498 Patent is four hours with effective drug delivery lasting only two hours. Film-style delivery devices with longer residence times and drug release profiles are not bioerodable or biodegradable meaning that a spent device must be physically removed from the site of application. Therefore, there is a need for a macroscopic, biodegradable or bioerodable drug delivery system capable of maintaining structural integrity and continuous drug release for a period of weeks for application in the oral, vaginal, or rectal cavity or periodontal pocket.

### SUMMARY OF THE INVENTION

The present invention relates to a system for the delivery of a therapeutic agent to the oral tissue, gum tissue, periodontal pocket, the surface of a tooth, a number of adjacent teeth, blood stream, or the buccal, rectal or vaginal mucus membranes, or the site of a epithelial lesion, or a combination thereof and more particularly to such composite delivery system wherein at least one surface layer comprises an agent selected from the group consisting of adhesive polymers, tackifying agents, and mixtures thereof, while at least one bulk layer comprises one or more therapeutic agents, bioerodable polymers, matrix polymers, or mixtures thereof. The surface layer may also include a therapeutic agent, which provides immediate delivery or “burst” of therapeutic agent to the treatment site. The bulk layer is formulated to provide a sustained release of the therapeutic agent. In combination, the delivery system provides both an initial rapid drug dose and a sustained delivery of the drug. In a preferred embodiment, the bulk layer comprises a mixture poly(lactic acid-co-glycolic acid) and ethyl cellulose.

The surface layer is designed to provide useful properties such as varying initial release of the active ingredient and/or adhesiveness, while the bulk layer that has limited swelling in human serum, gingival crevicular fluid, saliva, vaginal fluid or other fluids which bathe mucous membranes, provides for a sufficient and sustained release time. Even more particularly, the present invention relates to erodible delivery systems applied below gum line which are inserted at a dentist office, wherein such delivery systems have an effect for several weeks and are unobtrusive so as to be erodible with the flow of gingival crevicular fluid, saliva, or serum without surgical interference. The delivery system is capable of local delivery to the oral cavity or periodontal pocket or systemic delivery to the circulatory system. Alternative placements for the erodible delivery device include, the buccal, vaginal, rectal or other epithelial mucous membranes.

An alternative form of the invention includes an optional third, barrier layer which is impermeable to the therapeutic compound. This layer preferably comprises ethyl cellulose. In one embodiment, the barrier layer is placed on the opposite side of the device relative to the bioadhesive, surface layer such that the therapeutic is delivered preferentially to the mucous membrane contacted by the device. In another embodiment, the barrier layer is place between the bioadhesive layer and the bulk layer such that the therapeutic is delivered preferentially to the lumenal fluid, thus protecting the mucous membrane from direct contact with the therapeutic. In a variation of this embodiment, the adhesive layer itself is impermeable to the therapeutic of the bulk layer.

Exemplary therapeutic agents include antimicrobial agents, protease inhibitors, and anti-inflammatory agents, which are known in the art. Silver salts, and in particular, silver nitrate, are preferred antimicrobial agent for periodontal use.

### BRIEF DESCRIPTION OF THE DRAWINGS

FIG. 1 is a cross-sectional illustration of the composite drug delivery device of the invention.

FIG. 2 is a photomicrograph of a cross-section of a wafer of the invention.

FIG. 3 is a photomicrograph of the surface of the bulk layer of the wafer shown in FIG. 2.

FIGS. 4-6 are photomicrographs of the bulk layer of the wafer of FIG. 2; illustrating the progressive stages of degradation.

FIG. 7 is a plot of the cumulative release of silver ion over time from a bulk layer of the wafer of the invention.

FIG. 8 is a plot of total silver ion release in serum over time as measured by inductively coupled plasma (ICP).

FIG. 9 is a plot of cumulative release of silver ion based on a bioactivity assay.

FIG. 10 is a plot of percent swelling of bulk wafer of the invention in serum with time as a function of ethyl cellulose content.

FIG. 11 is a plot of cumulative release of benzylpenicillin from a bulk wafer of the invention.

FIG. 12 is a plot of force vs. time illustrating the improved adhesion in a composite wafer of the invention.

FIG. 13 is a plot of cumulative release of silver ion vs. time for the composite wafers of the invention.

FIG. 14 is a photomicrograph of a cross-section of the composite wafer of the invention illustrating an outer layer of starch and silver nitrate on the bulk layer.

FIG. 15 is a plot of load vs. time for the starch-coated composite wafers of Example 7.

FIG. 16 is a plot of cumulative silver ion release for the starch-coated composite wafers of Example 7.

FIG. 17 is a plot of percent swelling in serum for variously coated composite wafers of the invention.

### DETAILED DESCRIPTION

The present invention relates to a polymer system for the controlled delivery of bioactive materials, a solid composition for producing such a system, and a method for use of such a system in therapeutic treatment. The polymer system (wafer) has: (1) a bulk layer; (2) one or more optional surface layers and (3) an optional barrier layer. The polymer system of the present invention is advantageous in that it can be manipulated to control the amount of bioactive material released and the rate at which it is released. Further, it can be formulated to enhance retention of the device at the delivery site by the addition of a bioadhesive which adheres to mucous membranes and other moist surfaces. The present invention is also completely bioerodable, overcoming the discomfort and inconvenience of removing and reapplying a thin adhesive film.

The polymer composition is prepared by combining an active ingredient, a bioerodable polymer and/or a thermoplastic polymer to form a heterogeneous, solid polymeric matrix for the bulk layer in which the active ingredient is encapsulated by the bioerodable and/or thermoplastic polymer.

According to the invention, the bulk layer is formulated to provide a sustained release of the active ingredient(s) over a period of days, one, two, or three weeks or even months, as desired. The bulk layer may contain a single or multiple active ingredients which may be used to treat a single, or multiple conditions or diseases. Sustained release of the therapeutic(s) may be accomplished by using a combination of matrix polymers or by addition of a plasticizer to the bulk matrix. The polymer mixture or plasticizer is selected to modify the diffusion coefficient of the bulk so that the desired release profile is sustained. In preferred embodiments, the plasticizer provides greater solubility with the physiological fluids without an excessive amount of swelling. High levels of swelling are undesirable because it may increase patient pain and decrease retention time of the device, especially when placed in small areas like in the periodontal pocket.

The bulk layer of the composite delivery device may be made of one or more polymeric materials. Suitable bioerodable polymers for use in preparation of the interior bulk layer include gelatin, polyethylene glycol, polypropylene glycol, polyethylene oxide, copolymers of ethylene oxide and propylene oxide, copolymers of polyethylene glycol and polypropylene glycol, polytetramethylene glycol, polyether urethane, hydroxyethyl cellulose, ethyl cellulose, hydroxypropyl cellulose, hydroxypropylmethyl cellulose, alginate, collagen, polylactide, poly(lactide-co-glycolide) (PLGA), calcium polycarbophil, polyethymethacrylate, cellulose acetate, propylene glycol, polyacrylic acid, crosslinked polyacrylic acid, Carbopol™ (a commercially available form of polyacrylic acid), hydroxyethyl methacrylate/methyl methacrylate copolymer, silicon/ethyl cellulose/polyethylene glycol, urethane polyacrylate, polystyrene, polysulfone, polycarbonate, polyorthoesters, polyanhydrides, poly(amino acids), partially and completely hydrolyzed alkylene-vinyl acetate copolymers, polyvinyl chloride, polymers of polyvinyl acetate, polyvinyl alkyl ethers, styrene acrylonitrile copolymers, poly(ethylene terphthalate), polyalkylenes, poly(vinyl imidazole), polyesters and combinations of two or more of these polymers.

A particularly preferred embodiment includes a combination of PLGA and ethyl cellulose. PLGA is bioerodable and can be formulated to degrade over a wide range of conditions and rates. Ethyl cellulose is a water-insoluble polymer that can act as a plasticizer for the PLGA when a film is formed, but will be eroded in a bodily fluid. Due to its water-insolubility, it also has an effect on the degree and rate of swelling of the resultant film.

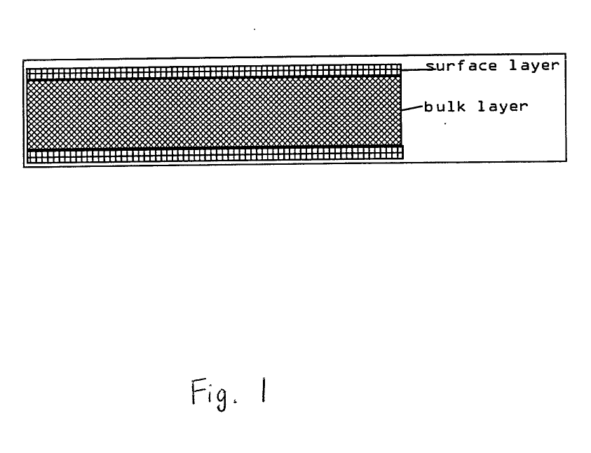

The surface layer(s) may be applied to one or both surfaces of the bulk layer and may include, in encapsulated or non-encapsulated form, the same or another active ingredient. The composite structure is schematically depicted in FIG. 1. Active ingredients include any component that causes a desired therapeutic effect at the delivery site, and may used to treat the same condition as treated by the compound of the bulk layer, or may be used to treat an entirely different condition. An unencapsulated active agent in the surface layer is responsible for rapid (“burst”) release of the active ingredient, while the bulk layer provides for the slow release.

Additional additives to the surface layer include adhesive agents, in particular mucoadhesive agents such as starch and diethylaminoehtyl-dextran (DEAE-dextran). A biodegradable mucoadhesive prolongs the residence time of the wafer at the site of application. Mucoadhesion overcomes the difficulty of prolonged exposure of wet, mucosal tissues to therapeutics which are normally washed away by bodily fluids. The vaginal, rectal and oral cavities have mucosal surfaces where direct and prolonged contact between a therapeutic and the epithelium may be desired. Adhesion to the site of an epithelial wound or lesion for prolonged therapeutic exposure is also desirable.

The present invention with an adhesive surface layer on one side can be used as a dressing to deliver therapy to internal and external wounds, including those caused by traumatic injury or surgery. Alternatively, a wafer with a mucoadhesive surface layer on both sides may be inserted into a laceration and used in place of, or in conjunction with sutures to bring the damaged tissue into a juxtaposition and promote healing. The bioerodable property of the wafer and the adhesive provides an advantage over conventional wound dressings which must be removed, often retraumatizing the injury site.

An optional, third layer that is impermeable to the therapeutic agent can also be added to the wafer. Preferably, the barrier layer is biodegradable or bioerodable. Preferable compositions for the barrier layer include ethyl cellulose, poly(acrylic acid) or other polyelectrolytes (e.g., chitosan) when the therapeutic of the bulk layer is hydrophilic. Hydrophobic modifications of polyelectrolytes provide effective barrier layers to hydrophobic therapeutics. In one embodiment of the wafer, the barrier layer is placed on the opposite side from the adhesive layer. This configuration has the effect of directing the released therapeutic agent onto the contacted epithelium rather than being diluted in the lumenal fluid. This configuration provides protection for the wafer from the lumenal fluid, increasing the life of the wafer and slowing the dissolution of the encapsulated medication.

In an alternative configuration of the wafer, the barrier layer is placed between the bulk layer and the adhesive layer. This configuration protects the underlying tissue from direct exposure to the medication, which is released into the lumen and diluted by the bodily fluid. This configuration can be used with highly concentrated forms of a therapeutic when delivery to the entire epithelial layer of the lumen is desired. The configuration is useful for therapeutics which are cytotoxic when administered at high concentrations. In an alternative form of this configuration, the mucoadhesive of the surface layer is an effective barrier, thus combining the functionality of the surface layer and the barrier layer into a single stratum.

The biologically active therapeutic compounds that may be loaded into the wafers of the present invention are any substance having biological activity, including proteins, polypeptides, polynucleotides, nucleoproteins, polysaccharides, glycoproteins, lipoproteins, and synthetic and biologically engineered analogs thereof.

Examples of biologically active compounds that might be utilized in a delivery application of the invention include literally any hydrophilic or hydrophobic biologically active compound. Preferably, though not necessarily, the drug is one that has already been deemed safe and effective for use by the appropriate governmental agency or body. For example, drugs for human use listed by the FDA under 21 C.F.R. 330.5, 331 through 361; 440-460; drugs for veterinary use listed by the FDA under 21 C.F.R. 500-582, incorporated herein by reference, are all considered acceptable for use in the present novel polymer networks.

The term “biologically active compound” includes pharmacologically active substances that produce a local or systemic effect in animals, plants, or viruses. The term thus means any substance intended for use in the diagnosis, cure, mitigation, treatment or prevention of disease or in the enhancement of desirable physical or mental development and conditions in an animal, plant, or virus.

The term “animal” used herein is taken to mean mammals, such as primates, including humans, sheep, horses, cattle, pigs, dogs, cats, rats, mice; birds; reptiles; fish; insects; arachnids; protists (e.g., protozoa); and prokaryotic bacteria.

The term “plant” means higher plants (angiosperms, gymnosperms), fungi, and prokaryotic blue-green “algae” (e.g., cyanobacteria).

The term “protein” is art-recognized and for purposes of this invention also encompasses peptides. The proteins or peptides may be any biologically active protein or peptide, naturally occurring or synthetic.

Examples of proteins include antibodies, enzymes, growth hormone and growth hormone-releasing hormone, gonadotropin-releasing hormone, and its agonist and antagonist analogues, somatostatin and its analogues, gonadotropins such as luteinizing hormone and follicle-stimulating hormone, peptide-T, thyrocalcitonin, parathyroid hormone, glucagon, vasopressin, oxytocin, angiotensin I and II, bradykinin, kallidin, adrenocorticotropic hormone, thyroid stimulating hormone, insulin, glucagon and the numerous analogues and congeners of the foregoing molecules.

Classes of pharmaceutically active compounds which can be loaded into responsive polymer network compositions of the invention include, but are not limited to, anti-AIDS substances, anti-cancer substances, antibiotics, immunosuppressants anti-viral substances, enzyme inhibitors, neurotoxins, opioids, hypnotics, antihistamines, tranquilizers, anti-convulsants, muscle relaxants and anti-Parkinson substances, anti-spasmodics and muscle contractants, miotics and anti-cholinergics, anti-glaucoma compounds, anti-parasite and/or anti-protozoal compounds, anti-hypertensives, analgesics, anti-pyretics and anti-inflammatory agents such as NSAIDs, local anesthetics, ophthalmics, prostaglandins, anti-depressants, anti-psychotic substances, anti-emetics, imaging agents, specific targeting agents, neurotransmitters, proteins, cell response modifiers, and vaccines, steroidal anti-inflammatory agents, bacteriocides, vasoconstrictors, chemotherapeutic drugs, antifingal drugs, vasodilator drugs, antiarrhythmic, antimigraine, peptide or protein drugs, antiasthmatics, and hemostatics.

A more complete listing of classes of compounds suitable for loading into polymers using the present methods may be found in the Pharmazeutische Wirkstoffe (Von Kleemann et al. (eds) Stuttgart/New York, 1987, incorporated herein by reference). Examples of particular pharmaceutically active substances are described below.

Anti-AIDS substances are substances used to treat or prevent Autoimmune Deficiency Syndrome (AIDS). Examples of such substances include 3′-azido-3+-deoxythymidine (AZT), 9-(2-hydroxyethoxymethyl)-guanine acyclovir, phosphonoformic acid, 1-adamantanamine, peptide T, and 2′,3′ dideoxycytidine.

Anti-cancer substances are substances used to treat or prevent cancer. Examples of such substances include methotrexate, cisplatin, prednisone, hydroxyprogesterone, medroxyprogesterone acetate, megestrol acetate, diethylstilbestrol, testosterone propionate, fluoxymesterone, vinblastine, vincristine, vindesine, daunorubicin, doxorubicin, hydroxyurea, procarbazine, aminoglutethimide, mechlorethamine, cyclophosphamide, melphalan, uracil mustard, chlorambucil, busulfan, carmustine, lomusline, dacarbazine (DTIC: dimethyltriazenomidazolecarboxamide), methotrexate, fluorouracil, 5-fluorouracil, cytarabine, cytosine arabinoxide, mercaptopurine, 6-mercaptopurine, and thioguanine.

Antibiotics are art recognized and are substances that inhibit the growth of or kill microorganisms. Antibiotics can be produced synthetically or by microorganisms. Examples of antibiotics include penicillin, tetracycline, chloramphenicol, minocycline, doxycycline, vancomycin, bacitracin, kanamycin, neomycin, gentamycin, erythromicin and cephalosporins, methicillin, oxacillin, cefalotin, cefaloridin, lincomycin, chlortetracycline, oxytetracycline, metacycline, streptomycin, gentamicin, and cycloserine.

Anti-viral agents are substances capable of destroying or suppressing the replication of viruses. Examples of anti-viral agents include a-methyl-P-adamantane methylamine, 1,-D-ribofuranosyl-1,2,4-triazole-3 carboxamide, 9->2-hydroxy-ethoxy!methylguanine, adamantanamine, 5-iodo-2′-deoxyuridine, trifluorothymidine, interferon, adenine arabinoside, protease inhibitors, thymadine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Enzyme inhibitors are substances that inhibit an enzymatic reaction. Examples of enzyme inhibitors include edrophonium chloride, N-methylphysostigmine, neostigmine bromide, physostigmine sulfate, tacrine HCl, tacrine, 1-hydroxy maleate, iodotubercidin, p-bromotetramisole, 10-(alpha-diethylaminopropionyl)-phenothiazine HCl, calmidazolium chloride, hemicholinium-3,3,5-initrocatechol, diacylglycerol kinase inhibitor I, diacylglycerol kinase inhibitor II, 3-phenylpropargylamine, N.sup.6 -monomethyl-L-arginine acetate, carbidopa, 3-hydroxybenzylhydrazine HCl, hydralazine HCl, clorgyline HCl, deprenyl HCl, L(−)-, deprenyl HCl, D(+)-, hydroxylamine HCl, iproniazid phosphate, 6-MeO-tetrahydro-9H-pyrido-indole, nialamnide, pargyline HCl, quinacrine HCl, semicarbazide HCl, tranylcypromine HCl, N,N-diethylaminoethyl-2,2-diphenylvalerate HCl, 3-isobutyl-1-methylxanthne, papaverine HCl, indomethacind, 2-cyclooctyl-2-hydroxyethylamine HCl, 2,3-dichloro-a-methylbenzylamine PCMB), 8,9-dichloro-2,3,4,5-tetrahydro-1H-2-benzazepine HCl, p-aminoglutethimide, p-aminoglutethimide tartrate,R(+)-, p-aminoglutethimide tartrate,S(−)-, 3-iodotyrosine, alpha-methyltyrosine, L-alpha-methyltyrosine, D,L-acetazolamide, dichlorphenamide, 6-hydroxy-2-benzothiazolesulfonamide, and allopurinol.

Neurotoxins are substances which have a toxic effect on the nervous system, e.g. nerve cells. Neurotoxins include adrenergic neurotoxins, cholinergic neurotoxins, dopaminergic neurotoxins, and other neurotoxins. Examples of adrenergic neurotoxins include N-(2-chloroethyl)-N-ethyl-2-bromobenzylamine HCl. Examples of cholinergic neurotoxins include acetylethylcholine mustard HCl. Examples of doparninergic neurotoxins include 6-hydroxydopamine HBr, 1-methyl-4-(2-methylphenyl)-1,2,3,6-tetrahydro-pyridine HCl, 1-methyl-4-phenyl-2,3-dihydropyridinium perchlorate, 1-methyl-4-phenyl-1,2,3,6-tetrahydropyridine HCl, 1-methyl-4-phenylpyridinium iodide.

Opioids are substances having opiate like effects that are not derived from opium. Opioids include opioid agonists and opioid antagonists. Opioid agonists include codeine sulfate, fentanyl citrate, hydrocodone bitartrate, loperamide HCl, morphine sulfate, noscapine, norcodeine, normorphine, thebaine. Opioid antagonists include nor-binaltorphimine HCl, buprenorphine, chlomaltrexamine 2HCl, funaltrexamione HCl, nalbuphine HCl, nalorphine HCl, naloxone HCl, naloxonazine, naltrexone HCl, and naltrindole HCl.

Hypnotics are substances which produce a hypnotic effect. Hypnotics include pentobarbital sodium, phenobarbital, secobarbital, thiopental and mixtures, thereof, heterocyclic hypnotics, dioxopiperidines, glutarimides, diethyl isovaleramide, a-bromoisovaleryl urea, urethanes, and disulfanes.

Antihistamines are substances which competitively inhibit the effects of histamines. Examples include pyrilamine, chlorpheniramine, tetrahydrazoline, diphenhydramine HCl, diphenhydramine salicylate, diphenhydramine HCl, chlorpheniramine maleate, isothipendyl HCl, tripelennamine HCl, promethazine HCl, and methdilazine HCl.

Tranquilizers are substances which provide a tranquilizing effect. Examples of tranquilizers include chloropromazine, promazine, fluphenzaine, reserpine, deserpidine, and meprobamate.

Anti-convulsants are substances which have an effect of preventing, reducing, or eliminating convulsions. Examples of such agents include primidone, phenytoin, valproate, and ethosuximide.

Muscle relaxants and anti-Parkinson agents are agents which relax muscles or reduce or eliminate symptoms associated with Parkinson's disease. Examples of such agents include mephenesin, methocarbomal, cyclobenzaprine HCl, trihexylphenidyl HCl, levodopa/carbidopa, and biperiden.

Anti-spasmodics and muscle contractants are substances capable of preventing or relieving muscle spasms or contractions. Examples of such agents include atropine, scopolamine, oxyphenonium, and papaverine.

Miotics and anti-cholinergics are compounds which cause bronchodilation. Examples include echothiophate, pilocarpine, physostigmine salicylate, diisopropylfluorophosphate, epinephrine, neostigmine, carbachol, methacholine, and bethanechol.

Anti-glaucoma compounds include betaxalol, pilocarpine, timolol, timolol salts, and combinations of timolol, and/or its salts, with pilocarpine.

Anti-parasitic, -protozoal and -fungals include ivermectin, pyrimethamine, trisulfapyrimidine, clindamycin, amphotericin B, nystatin, flucytosine, natamycin, and miconazole.

Anti-hypertensives are substances capable of counteracting high blood pressure. Examples of such substances include alpha-methyldopa and the pivaloyloxyethyl ester of alpha-methyldopa, amlodipine, benazepril HCl, captopril, clonidine HCl, diazoxide, diltiazem HCl, enalapril maleate, enalaprilat, felodipine, isradipine, nicardipine HCl, nifedipine, atenolol, metroprolol tartarate, oxpenolol HCl, propanolol HCl and verapamil HCl.

Analgesics are substances capable of preventing, reducing, or relieving pain. Examples of analgesics include morphine sulfate, codeine sulfate, meperidine, and nalorphine.

Anti-pyretics are substances capable of relieving or reducing fever and anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide, acetaminophen, methyl salicylate, monoglycol salicylate, mefenamic acid, flufenamic acid, alclofenac, diclofenac sodium, ibuprofen, ketoprofen, naproxen, pranoprofen, fenoprofen, sulindac, fenclofenac, clidanac, flurbiprofen, fentiazac, bufexarnac, piroxicam, phenylbutazone, oxyphenbutazone, flofezone, pentazocine, mepirizole, and tiaramide HCl.

Local anesthetics are substances which have an anesthetic effect in a localized region. Examples of such anesthetics include procaine, lidocain, tetracaine, dibucaine, dibucaine HCl, lidocaine HCl, benzocaine, p-buthylaminobenzoic acid 2-(diethylamino) ethyl ester HCl, procaine HCl, tetracaine HCl, chloroprocaine HCl, oxyprocaine HCl, mepivacaine, cocaine HCl, and piperocaine HCl, dyclonine, and dyclonine HCl.

Ophthalmics include diagnostic agents such as sodium fluorescein, rose bengal, methacholine, adrenaline, cocaine, and atropine. Ophthalmic surgical additives include alpha-chymotrypsin and hyaluronidase.

Prostaglandins are art recognized and are a class of naturally occurring chemically related, long-chain hydroxy fatty acids that have a variety of biological effects.

Anti-depressants are substances capable of preventing or relieving depression. Examples of anti-depressants include imipramine, amitriptyline, nortriptyline, protriptyline, desipramine, amoxapine, doxepin, maprotiline, tranylcypromine, phenelzine, and isocarboxazide, cluoxetine HCl, maprotiline HCl, and phenelzine sulfate.

Anti-psychotic substances are substances which modify psychotic behavior. Examples of such agents include phenothiazines, butyrophenones and thioxanthenes.

Anti-emetics are substances which prevent or alleviate nausea or vomiting. An example of such a substance includes Dramamine®, metoclopramide HCl, nabilone, and ondansetron HCl..

Imaging agents are agents capable of imaging a desired site, (e.g. tumor) in vivo. Examples of imaging agents include substances having a label, which is detectable in vivo, such as antibodies attached to fluorescent labels. The term antibody includes whole antibodies or fragments thereof.

Specific targeting agents include agents capable of delivering a therapeutic agent to a desired site, e.g. tumor, and providing a therapeutic effect. Examples of targeting agents include agents that can carry toxins or other agents that provide beneficial effects. The targeting agent can be an antibody linked to a toxin like ricin A or an antibody linked to a drug.

Neurotransmitters are substances that are released from a neuron on excitation and travel to either inhibit or excite a target cell. Examples of neurotransmitters include dopamine, serotonin, gamma-aminobutyric acid, norepinephrine, histamine, acetylcholine, and epinephrine.

Steroidal anti-inflammatory agents include hydrocortisone, prednisolone, dexamethasone, triamcinolone acetonide, fluocinolone acetonide, hydrocortisone acetate, prednisolone acetate, methylprednisolone, dexamethasone acetate, betamethasone, betamethasone valerate, flumetasone, fluorometholone, budesonide, and beclomethasone dipropionate.

Bacteriocides and disinfectants include thimerosol, phenol, thymol, benzalkonium chloride, benzethonium chloride, chlorhexidine, providone iode, cetylpyridinium chloride, eugenol, and trimthylammonium bromide.

Vasoconstrictors include naphazoline nitrate, tetrahydrozoline HCl, oxymetazoline HCl, phenylephire HCl, and tramazoline HCl.

Hemostatics include thrombin, phytonadione, protamine sulfate, aminocaprioc acid, tranexamic acid, carbaxochrome, carbaxochrome sodium sulfonate, rutin, and hesperidin.

Chemotherapeutic drugs include vinblastine, cis-platin, 5-fluorouracil (5FU), methotrexate, 6 mercaptopurine, 1-beta-D-arabinofuranosylcytosine, mechlorethamine, chlorambucil, melphalan oxazaphosphorines, carboplatin, JM40, spiroplatin, tetraplatin, JM216, taxol, sulfamide, sulfathiazole, sulfadiazine, homosulfamine, sulfisoxazole, sulfisomidine, sulfamethizole, and nitrofurazone.

Antifungal drugs include amphotericin, clotrimazole, econazole nitrate, fluconazole, griseofulvin, itraconazole, ketoconazole, niconazole, griseofulvin, itraconazole, ketoconazole, miconazole, nystatin, terbinafine HCl, undecenoio acid, and zinc undecenoate.

Vasodilator drugs include buflomedil HCl, bupheneine HCl, oxpentifylline, glyceryl trinitrate, isosorbide dinitrate, isosorbide mononitrate and pentaerythritol tetranitrate.

Antiarrhythmic drugs include quinidine, quinidine salts, procainamide hydrochrolide, lidocaine, and mexiletine HCl.

Antimigraine drugs include dihydroergotamine mesylate, ergotamine tartarate, sumatriptan succinate and other triptan drugs.

Peptide or protein drugs include insulin, buserelin acetate, goserlin acetate, leuprorelin acetate, calcitonin, cyclosporin, gonadorelin, somastatin, vasopressin, oxytocin, interferon, and human growth hormone.

Antiasthmatics include salbutamol and terbutaline sulfate.

Cell response modifiers are chemotactic factors such as platelet-derived growth factor (PDGF). Other chemotactic factors include neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, nerve growth factor, and bone growth/cartilage-inducing factor (alpha and beta), or other bone morphogenetic protein.

Other cell response modifiers are the interleukins, interleukin inhibitors or interleukin receptors, including interleukin 1 through interleukin 10; interferons, including alpha, beta and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, and activin; and bone morphogenetic proteins.

The composite delivery system may include those additives and excipients that are known in the art.

In preferred embodiments, the device is a non-deformable, or more preferably a deformable, film at room and body temperatures (20° C. to 30° C.). The deformable film can be placed in the periodontal pockets, adjacent to the exposed tooth roots and/or alveolar bone during periodontal procedures. Such placement allows contact between the film and the periodontal-disease causing microorganisms. In other preferred embodiments, the film can be placed on the epithelium of the oral, vaginal or rectal cavities or on a dermal wound or lesion.

The invention is illustrated by the following examples.

### EXAMPLE 1

Method of fabrication of composite periodontal wafer for administration below gum line

Periodontal wafers were prepared from finely powdered (particle size below 10 μm) poly(D,L-lactic-co-glycolic) acid (PLGA, lactide:glycolide molar ratio, 50:50, obtained B. I. Chemicals, Inc., Wallingford, Conn., as Resomer RG 502H), ethyl cellulose NF premium obtained from Dow Chemical (Lot#ME05013TO1, trade name ETHOCEL) and AgNO₃ (USP grade, obtained from Spectrum Chemical Corp., Gardena, Calif.) mixture by a compression-molding technique. Herein, silver nitrate is an active antimicrobial agent, PLGA is a bioerodable thermoplastic polymer, and ethyl cellulose is a water-insoluble thermoplastic polymer. An appropriate powder blend was placed into a Teflon-coated steel mold assembly with a 50×50×0.5 mm opening and pressed for 60 s at 70° C. and 12,500 psi using a Carver Model 3912 hydraulic unit (Carver, Inc., Wabash, Ind.). The assembly was allowed to equilibrate at ambient temperature and the resultant film was extracted from the mold and was stamped at 60° C. to yield wafers of 4×5×0.5 mm or 2×4×0.5 mm size. The wafers were stored at −20° C. in sealed foil pouches. Scanning electron microscopy (SEM) was used to observe the structure of the resulting wafers and qualitative changes in wafer morphology upon degradation in serum over time. The samples were kept in human serum at 37° C., withdrawn, gently washed with water, snap-frozen and lyophilized. Human serum resembles composition of gingival crevicular fluid (GCF) abundant in periodontal pocket. Human serum from clotted male whole blood was obtained from Sigma Chemical Co. (St. Louis, Mo.) and used to model GCF that in fact is an altered serum transudate found in the gingival sulcus [F. A. Scannapieco, M. J. Levine, Saliva and dental pellicles, In: Contemporary Periodontics, R. J. Genco, H. M. Goldman, D. W. Cohen, Eds., The C. V. Mosby Co., St. Louis, Mo., 1990, pp.117-125]. The dried samples were sputter-coated with gold 200-300 A and microscopy was performed with a dual-stage electron microscope DS-701 (Topcon Co., Tokyo, Japan). To visualize the cross-section of the wafer structure, the wafers were freeze-fractured using snap-freezing in liquid nitrogen.

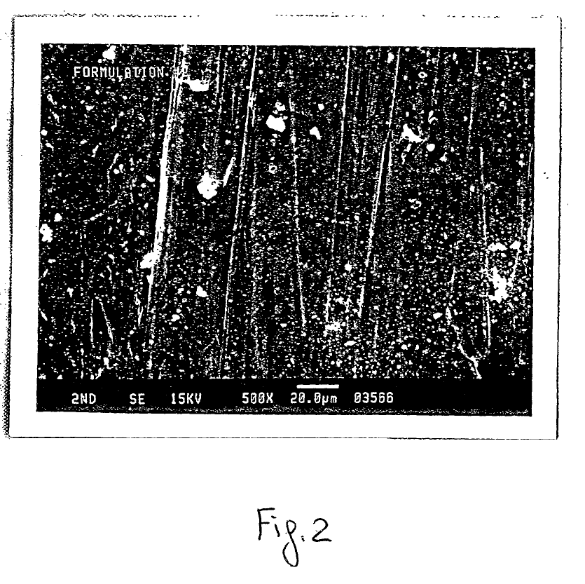

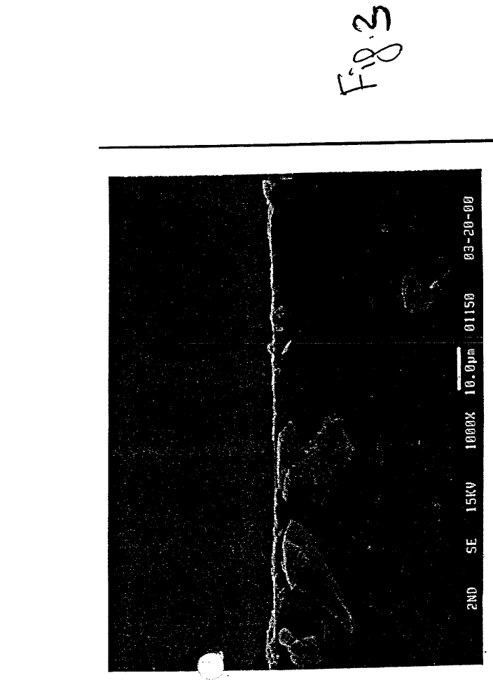

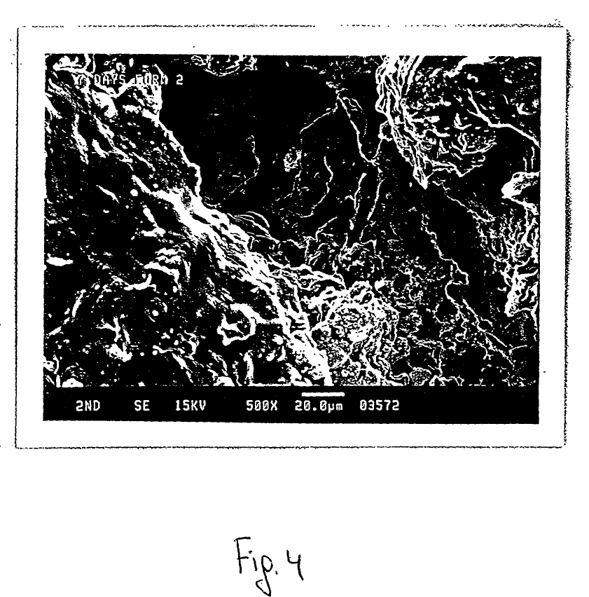

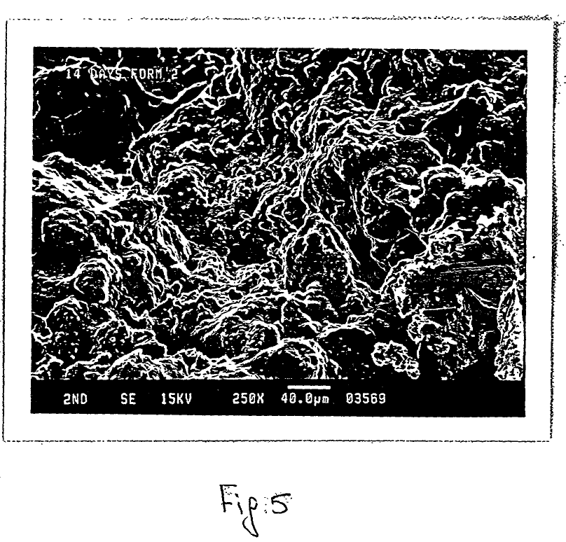

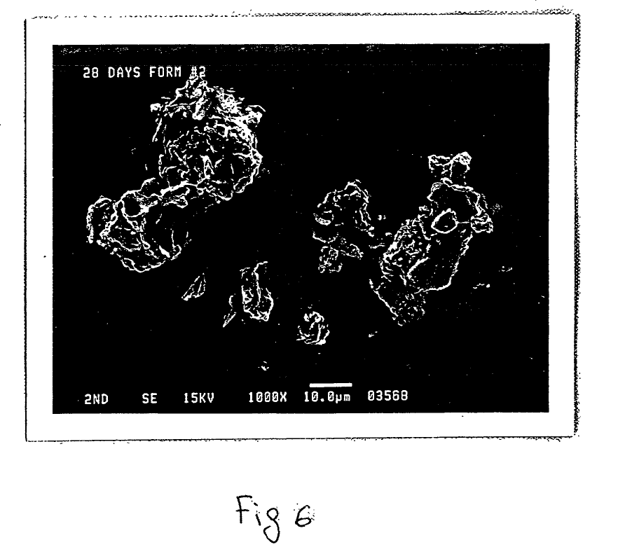

FIGS. 2 and 3 show microphotographs of the cross-section and the surface of the wafers composed of 61.9% PLGA, 14.2% ethyl cellulose, and 23.9% AgNO₃ made by the above technique. As is seen, the surface of the wafers was smooth and contained exposed crystals of AgNO₃ (bright spots in FIG. 2). Both fully embedded and partially exposed particles of the active ingredient (silver nitrate) are observed in the surface and the bulk layer, respectively (FIG. 3). These observations confirm heterogeneous structure of the obtained wafers. Degradation of the wafers in human serum is illustrated in FIGS. 4-6. Wafers kept in serum for 28 days eventually transformed into irregularly shaped particles of ethyl cellulose of 1-5 μm effective size (FIG. 6). The PLGA initially served to bind ethyl cellulose and silver nitrate particles into a robust wafer (compare with FIG. 2) fully degraded and eroded, while silver nitrate dissolved in serum. These observations confirm that the prolonged exposure of the periodontal wafers to the bodily fluids results in degradation and erosion of the wafers leaving small particles that are moved in the oral cavity by the hydrodynamic flows.

### EXAMPLE 2

Sustained release of the active ingredient from periodontal wafers

Periodontal wafers were fabricated as in Example 1 by compression-molding technique. Wafers consisted of 24% silver nitrate, and specified content of ethyl cellulose and PLGA. The release of silver from a periodontal wafer was measured in vitro, using a wafer of a known initial weight. The experimental procedure involved placement of the wafer in a vial with 1.0 mL of either deionized water or human serum from clotted male whole blood (Sigma Chemical Co., St. Louis, Mo.) followed by incubation at 37° C. Wafers placed in serum were continuously shaken at 250 rpm using a KS 10 orbital shaker (BEA-Enprotech Corp., Hyde Park, Mass.) in an environmental chamber at 37° C. The release medium was intermittently withdrawn from the vials and assayed for silver content, while fresh medium was added to the vials. Silver content in water was measured against standard curves using an Orion Model 290A pH/ISE Meter equipped with a Model 9416 Silver/Sulfide Electrode (Orion Research, Inc., Beverly, Mass.). Total silver content in serum was assayed using a Spectroflame ICP Model FMD-07 inductively coupled plasma atomic emission spectrometer (Spectro Inc., Littleton, Mass.). The frequency of the sample withdrawal was chosen to ensure that the sink conditions were maintained, such that the liquid sample surrounding the wafer contained less than 10% of the initial silver load.

Silver released from the wafer was assayed for biological activity using E. coli containing the impA-mutation [B. A Sampson, R. Misra, S. A. Benson, Identification and characterization of a new gene of Escherichia coli K-12 involved in outer membrane permeability, Genetics, 1989, 122:491-501], which makes the cells particularly susceptible to silver toxicity. The assay is based on incubating cells in the presence of two-fold dilutions of samples and testing for growth inhibition. Cells were incubated on a Brain Heart Infusion (BHI) agar plate (Binax/Nel, Waterwille, Me.) overnight at 37° C. The cells were resuspended in cation-adjusted Mueller-Hinton growth medium (CAMH) (Becton Dickinson Bioscience, Sparks, Md.). The absorbance at 600 nm was determined, and the cell suspension was diluted in CAMH. An aliquot (50 μL) of the cell suspension was added to each well of a microtiter tray already containing 50 μL of a sample diluted into CAMH. The cells were incubated for 5 hours at 37° C. The last dilution that inhibits growth is compared to the last dilution that inhibits growth when a standard silver nitrate solution is similarly tested. CAMH medium may interact with silver ion, therefore the calibration curve was developed in the same medium.

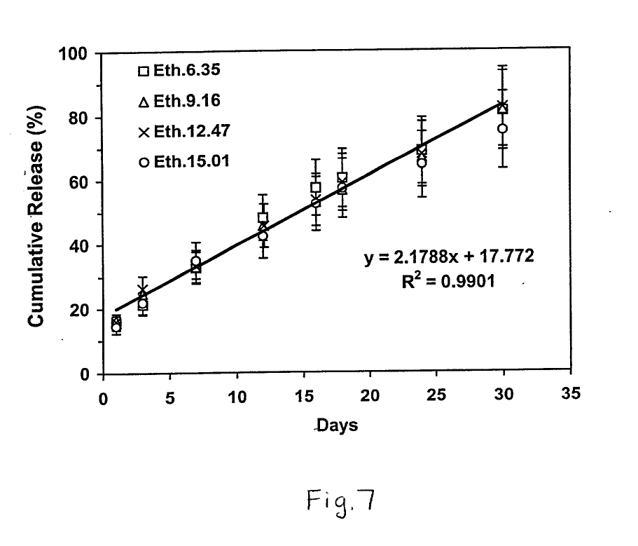

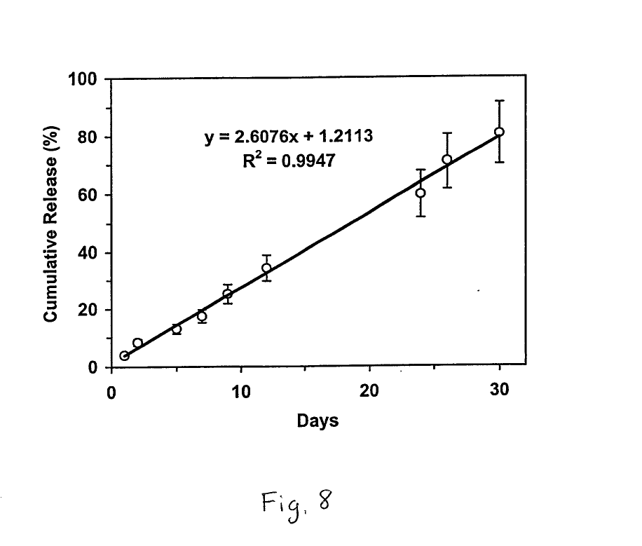

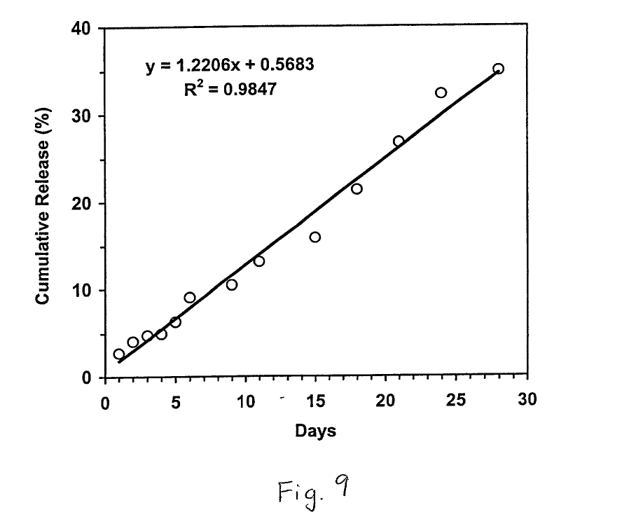

The results of the release in water are presented in FIG. 7 and indicate content of ethylcellulose in the wafers as a percentage of total wafer weight. Diffusion—controlled release with a linear (zero-order kinetic) profile was obtained for matrix-forming agent, ethyl cellulose at 6-15%. A linear release profile is the most desirable in controlled release applications because it results in a constant dose delivery over time. Release of total silver in serum measured by ICP is presented in FIG. 8. The wafer contents were PLGA, 67%; ethyl cellulose, 9%; silver nitrate, 24%. Again, linear (zero-order) release was observed within a period of over 30 days. Initial “burst” was low demonstrating effectiveness of the silver encapsulation. The linearity of the release indicates that diffusion from the matrix of interconnected ethylcellulose particles dominates over degradation of the PLGA. Cumulative release of silver in serum based on bioactivity assay is presented in FIG. 9. It can be seen that zero-order rate of supply of antimicrobial silver was maintained over 30 days. These results are more favorable than in any known subgingival drug delivery system.

### EXAMPLE 3

Swelling of periodontal wafers in serum

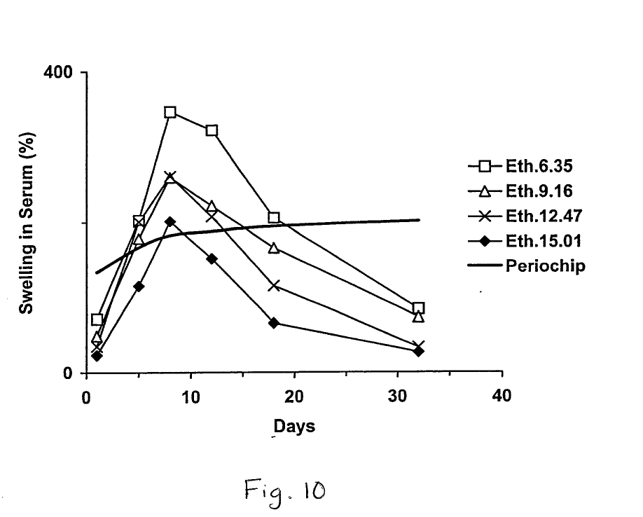

Water uptake by the periodontal wafers affects their retention time, as excessive swelling may lead to the exposure of the wafer outside of the periodontal pocket. Therefore, it is necessary to optimize changes in volume/mass of the wafers allowed to degrade/erode in serum or gingival crevicular fluid. Wafers fabricated as in Example 1 were subjected to the study of swelling/mass loss. The swelling (S) of the wafers placed in serum was measured by periodical weighing and expressed by S, %=100×(W_{S}/W_{O}−1). Here, W_{S} and W_{O} is the swollen and initial weight of the wafer, respectively. FIG. 10 shows the swelling of wafers in serum at 37° C. as a function of ethylcellulose concentration. Initial AgNO₃ content is 12%. PLGA concentration is varied. Numbers designate initial ethyl cellulose concentrations. Solid line represents swelling of commercially available PerioChip®. Error bars (15-18%) are not shown for clarity. Swelling of the wafers in serum decreased with the content of the water-insoluble ethyl cellulose and was comparable to that of the commercially available PerioChip® consisting of a cross-linked gelatin matrix. These results provide means of optimizing the mass uptake/retention time of the periodontal wafers by varying contents of water-insoluble (but dispersible) thermoplastic polymer.

### EXAMPLE 4

In vitro release of benzylpenicillin from periodontal wafers

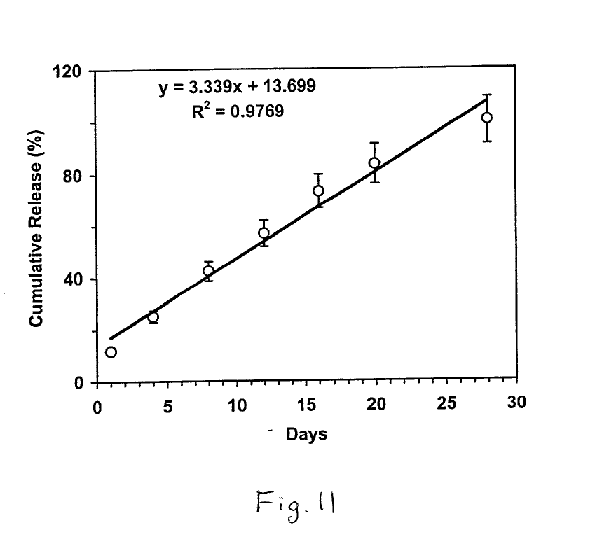

This example demonstrates zero-order release of antimicrobial agents other than metal ions from the heterogeneous periodontal wafer. The wafers were fabricated as described in Example 1, except benzylpenicillin (Sigma, Penicillin-G, potassium salt; Lot#84H1016, 1575 units per mg) was used as an active ingredient. The wafer contents were PLGA, 79%; benzylpenicillin, 9%; ethyl cellulose, 12%. Benzylpenicillin in water was assayed fluorometrically. Fluorescence spectra were recorded using a 10-mm path length quartz cell using a Shimadzu Model RF-5301 PC spectrofluorophotometer (slit widths 10 nm) at right-angle geometry. Emission spectra were recorded (λex=280 nm) and peak intensity (λem=310 nm) was plotted versus concentration to develop appropriate calibration curves. The release of benzylpenicillin in water at 37° C. is shown in FIG. 11. A zero-order release was demonstrated over 30 days.

### EXAMPLE 5

Fabrication of adhesive wafer of composite structure via compression-molding technique

Enhancement of adhesive characteristics of the heterogeneous wafers is described. The enhancement is achieved by coating the surfaces of the wafers with layers of adhesive polymers.

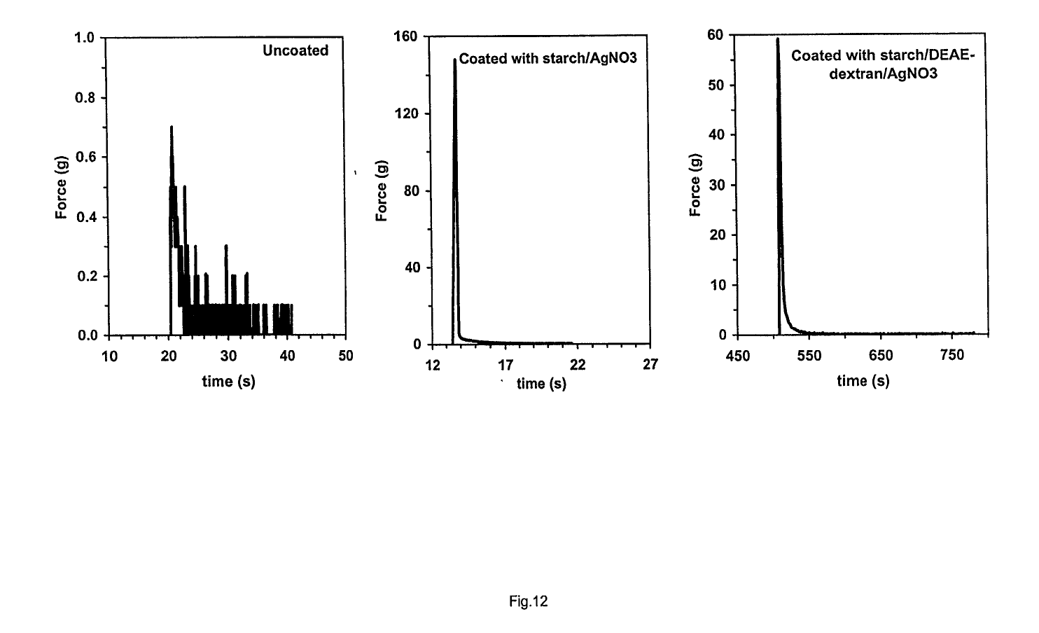

To prepare adhesive wafers, heterogeneous wafer films of about 5×5×0.05 cm were fabricated as described in Example 1. The film contents were PLGA, 67%; ethyl cellulose, 9%; AgNO₃, 24%. The film was then put onto a layer of the powder made of (i) a blend of diethylaminoethyl-dextran (DEAE-dextran) and starch (1:1 w/w); (ii) a blend of starch and AgNO₃ (1:1 w/w); (iii) a blend of starch, DEAE-dextran, and AgNO₃ (1:1:1 w/w). The film was then covered with identical layer of corresponding powder and pressed at 165° F. and 12500 psi. The resulting composite structure was 0.5 mm thick and its surfaces were coated with layers of bioadhesive material. In order to test the adhesive properties of the resulting wafers, the latter were tested for their adhesive characteristics using a TA.XT2I Texture Analyzer (Texture Technologies Corp.). The wafers were attached to a probe using double-sided film and tested for adhesion on a polished surface of the calf tooth wet with serum. The results are shown in FIG. 12. Unmodified wafer showed a nominal maximum detachment force of 0.769 g. Under identical conditions, wafers with surfaces modified with starch/AgNO₃ and starch/DEAE/AgNO₃ blends exhibited maximum detachment force of 23.81 and 61.46 g, respectively, at least 30-and 80-fold improvement in adhesion, respectively.

### EXAMPLE 6

In vitro release from adhesive wafer of composite structure fabricated via compression-molding technique

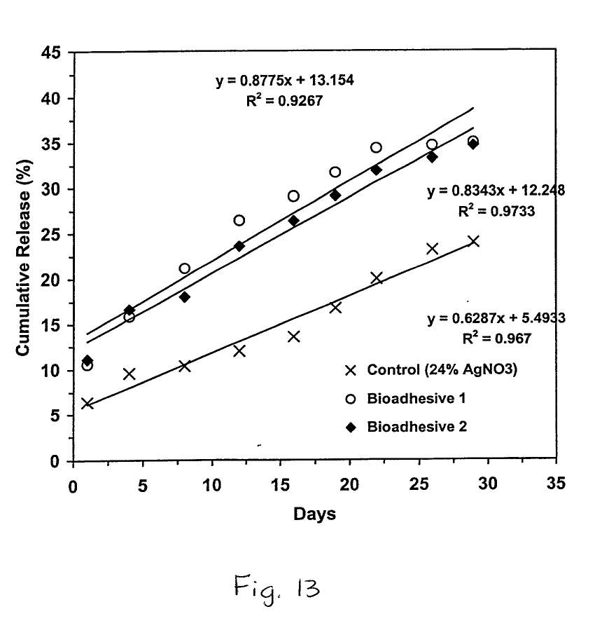

Adhesive wafers fabricated as described in Example 5 were subjected to the silver release studies. Cumulative release of silver in serum based on bioactivity assay is presented in FIG. 13. Uncoated wafers prepared as in Example 1 are designated Control, whereas wafers coated with AgNO₃/starch and AgNO₃/DEAE-dextran/starch are designated bioadhesive 1 and bioadhesive 2, respectively. Useful rapid release of the antimicrobial agent over the first 1 day was observed with the adhesive wafers. It can be seen that linear supply of antimicrobial silver was maintained over 30 days in all cases.

### EXAMPLE 7

Fabrication of adhesive wafer of composite structure via wet-coating technique

Further enhancement of adhesive characteristics of the heterogeneous wafers in described. The enhancement is achieved by coating the surfaces of the wafers with layers of adhesive polymers.

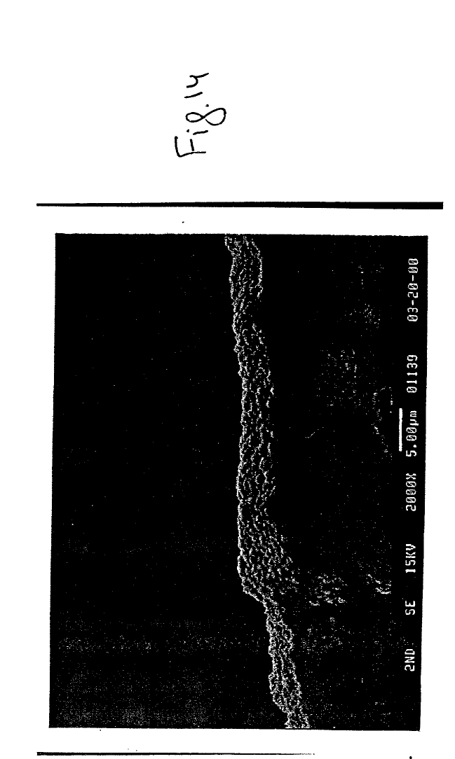

To prepare adhesive wafers, first heterogeneous wafer films of about 5×5×0.05 cm were fabricated as described in Example 1. The film contents were PLGA, 67%; ethyl cellulose, 9%; AgNO₃, 24%. The films were then coated by dipping into various solutions (Table 1) for 1 min followed by drying the wafers on air and lyophilization overnight. The content of dipping solution and the resulting wafers are given in Table 1. Structure of the coated wafers was observed using scanning electron microscopy after-freeze-fracturing in liquid nitrogen as described in Example 1. The wafers had a 2-5 μm thick layer of grain-like particles of starch/AgNO₃ on both surfaces, while the Bulk of the wafers showed irregularly shaped large AgNO₃ particles embedded into the Ethocel/PLGA melt upon compression (FIG. 14). Uncoated wafers (FIG. 3) had larger particles embedded with some particles seen on the surface. The SEM study clearly shows the composite structure of the coated wafers.

### EXAMPLE 8

Adhesive properties of the composite wafers fabricated via wet-coating technique

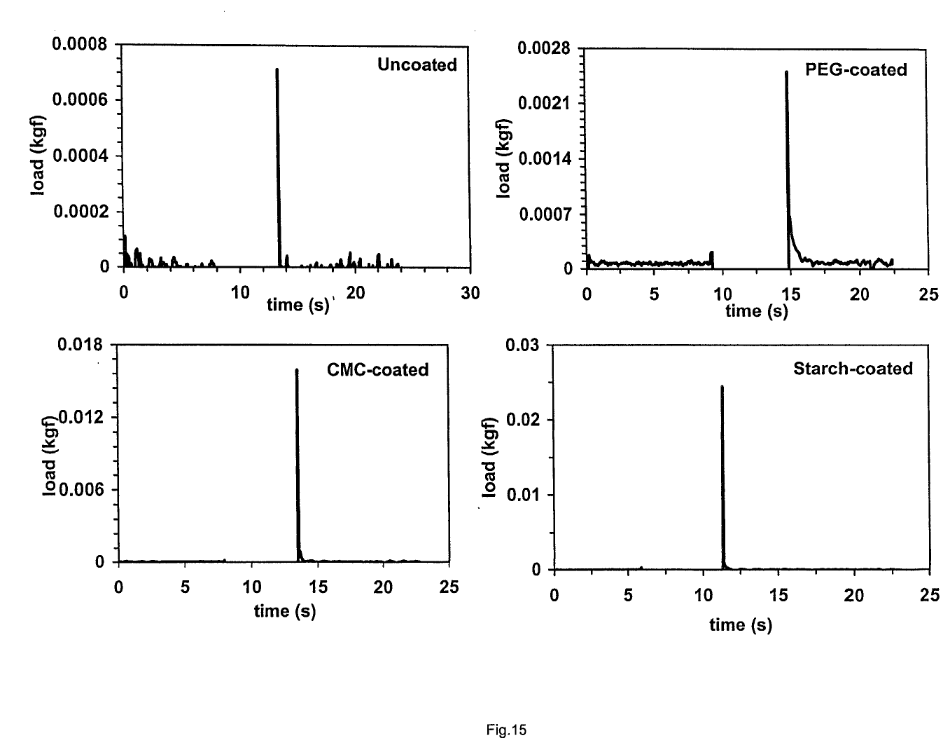

Testing of the wafers was conducted using a Model 5542 Single Column Materials testing System with a high performance loading frame (Instron Co., Canton, Mass.). Wafer of about 4×4×0.5 mm size was loaded onto a polished calf tooth wet with serum. The wafer was held at tooth surface for 10 s with a force of 200 g and then a detachment force was measured using programmable loading frame. It was shown that the wafers coated with adhesive compositions possessed up to 37-fold higher maximum detachment force (measure of adhesion) compared to parent uncoated wafers (FIG. 15). Starch/AgNO3-coated wafers exhibited the maximum adhesion toward the teeth. Therefore, such coated periodontal wafers possess capability of improved retention when inserted in periodontal pocket.

### EXAMPLE 9

Release and swelling characteristics of the composite wafers fabricated via wet-coating technique

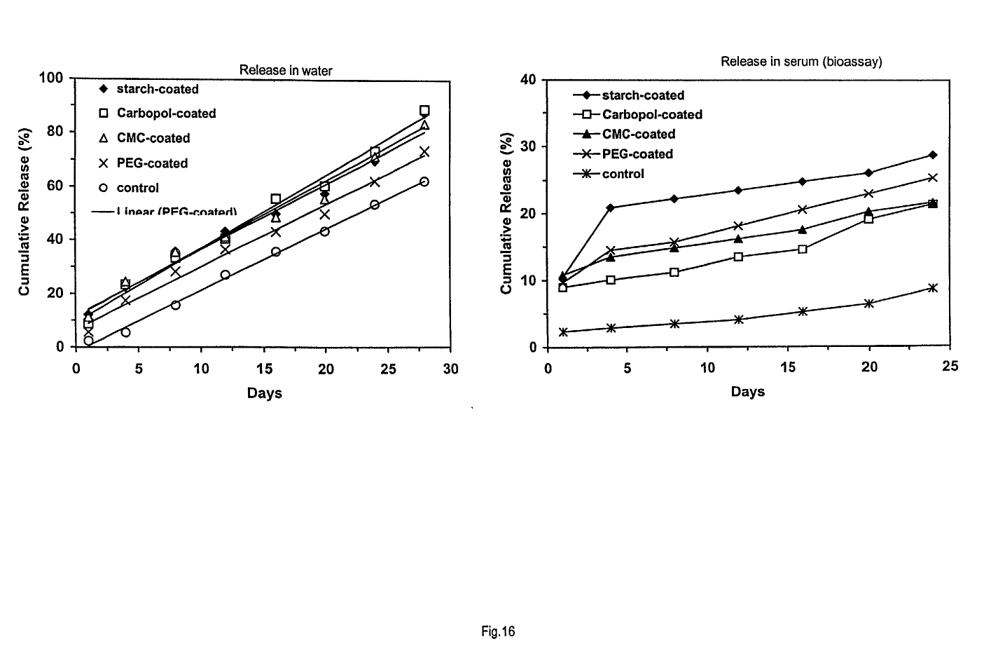

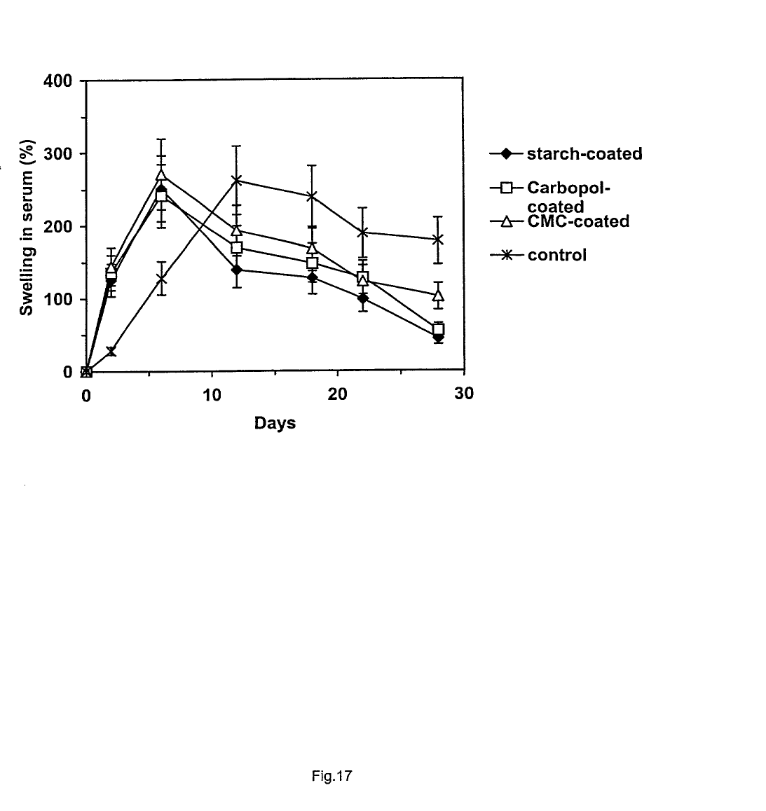

Composite wafers fabricated as described in Example 7 were subjected to the silver release studies. Cumulative release of silver in water and in serum (based on bioactivity assay) is presented in FIG. 16. Uncoated wafers prepared as in Example 1 are designated control, whereas wafers coated as described in Table 1 are designated as coated. Zero-order release was observed in water for over 30 days in all cases. Useful enhancement of bioactivity of coated wafers was observed, in particular with wafers coated with starch and AgNO₃ layers. Swelling in serum at 37° C. is presented in FIG. 17. Modest mass uptake by the coated wafers, in combination with their demonstrated adhesive properties, indicates that they would possess high retention in periodontal pocket.

TABLE 1
Compositions used for coating of heterogeneous wafers.

| | | | | | Wt. % | Wt % |
|---|---|---|---|---|---|---|
| | | Wt. | Wt | | AgNO₃ | AgNO₃ |
| | | Before | After | Wt. % | in | in |
| No. | System | dip | Dip | Coating | coating | wafer |
| 1 | 500 mg | 142 | 150.8 | 6.1971 | 2.05 | 25.4 |
| | NaCMC type | | | 83099 | | |
| | 7L2P, | | | | | |
| | Lot 68922 | | | | | |
| | (Hercules) | | | | | |
| | 250 mg AgNO₃ | | | | | |
| | 1 mL water | | | | | |
| | 1 mL acetone | | | | | |
| | (9% AgNO₃) | | | | | |
| 2 | 500 mg | 599 | 625 | 4.34 | 1.43 | 25.3 |
| | Corn Starch | | | | | |
| | Sigma S-9679, | | | | | |
| | Lot 49HO568 | | | | | |
| | 250 mg AgNO₃ | | | | | |
| | 1 mL water | | | | | |
| | 1 mL acetone | | | | | |
| 3 | 500 mg | 85 | 87 | 2.35 | 0.776 | 25.09 |
| | Carbopol | | | | | |
| | 971P-NF | | | | | |
| | Lot | | | | | |
| | CC76AAJO22 | | | | | |
| | (BFGoodrich) | | | | | |
| | 250 mg AgNO₃ | | | | | |
| | 10 mL water | | | | | |
| | 1 mL acetone | | | | | |
| | 0.5 ml NaOH | | | | | |
| | (0.1 N) | | | | | |
| 4 | MELT of | 121 | 164 | 35.5 | 0 | 16.05 |
| | polyglycol | | | | | |
| | E900NF Lot | | | | | |
| | KI1601N7PA | | | | | |
| | (Dow | | | | | |
| | Chemical) | | | | | |
| | at 70° C. | | | | | |

What is claimed is:

1. A bioerodable pharmaceutical carrier device comprising: (i) at least one bulk layer designed to release a therapeutic agent over a prolonged period of time where said bulk layer comprises a therapeutic agent and a bioerodable matrix polymer wherein said polymer comprises a combination of a thermoplastic polymer and a water insoluble plasticizer; (ii) a surface layer on one or more sides of the bulk layer consisting of a biodegradable mucoadhesive agent; (iii) an bioerodable, impermeable barrier layer.

2. The composition of claim 1, wherein said barrier layer is between said bulk and said surface layers.

3. The composition of claim 1, wherein said bulk layer is between said barrier and said surface layers.

4. The composition of claim 1, wherein said surface layer further comprises a therapeutic agent.

5. The composition of claim 1, wherein said bulk layer comprises a combination of a poly(lactic acid-co-glycolic acid) (PGLA) polymer and the ethyl cellulose.

6. The composition of claim 5, wherein said mucoadhesive is either starch, diethylaminoethyl-dextran (DEAE-dextran), or a mixture of (DEAE-dextran) and starch.

7. The composition of claim 6, wherein said therapeutic is an antimicrobial agent.

8. The composition of claim 7, wherein said antimicrobial agent is silver nitrate.

9. The composition of claim 7, wherein said antimicrobial agent has antiviral properties.

10. The composition of claim 7, wherein said antimicrobial agent has antifungal properties.

11. The composition of claim 6, wherein said therapeutic is an anti-inflammatory agent.

12. The composition of claim 6, wherein said therapeutic is a hemostatic agent.

13. The composition of claim 6, wherein said therapeutic is a chemotherapeutic agent.

14. A bioerodable pharmaceutical carrier device comprising: (i) a bulk layer comprising a therapeutic agent and a mixture of poly(lactic acid-co-glycolic acid) (PGLA) and ethyl cellulose; (ii) a surface layer on one or more sides of the bulk layer comprising a biodegradable mucoadhesive agent.

15. The composition of claim 14, wherein said surface layer further comprises a therapeutic agent.

16. The composition of claim 14, wherein said mucoadhesive is either starch, diethylaminoethyl-dextran (DEAE-dextran), or a mixture of (DEAE-dextran) and starch.

17. The composition of claim 14, wherein said therapeutic is an antimicrobial agent.

18. The composition of claim 17, wherein said antimicrobial agent is silver nitrate.

19. The composition of claim 17, wherein said antimicrobial agent has antiviral properties.

20. The composition of claim 17, wherein said antimicrobial agent has antifungal properties.

21. The composition of claim 15, wherein said therapeutic is an anti-inflammatory agent.

22. The composition of claim 15, wherein said therapeutic is a hemostatic agent.

23. The composition of claim 15, wherein said therapeutic is a chemotherapeutic agent.

24. A method of treating or preventing periodontal disease in a human patient by placing the composition of claim 17 within the oral cavity.

25. The method of claim 24, wherein said device is placed in the periodontal pocket.

26. The method of claim 24, wherein said device is placed below the gum line.

27. The method of claim 24, wherein said device is placed on the buccal epithelium.

28. The method of claim 24, wherein said antimicrobial agent is silver nitrate.

29. A method of treating an infection of mucous membrane epithelium in a human patient comprising administering to said patient the composition of claim 14, wherein said device is placed on the affected mucous membrane.

30. The method of claim 29, wherein said therapeutic is an antiviral agent.

31. The method of claim 30, wherein said infection is caused by a virus.

32. The method of claim 31, wherein said infection is by the human papaloma virus.

33. The method of claim 31, wherein said infection is of the buccal epithelium.

34. The method of claim 31, wherein said infection is of the vaginal epithelium.

35. The method of claim 31, wherein said infection is of the rectal epithelium.

36. The method of claim 29, wherein said infection is of a dermal or epidermal tissue.
